(19) United States
(12) Patent Application Publication
(10) Pub. No.: US 2003/0100024 A1
(43) Pub. Date: May 29, 2003

(54) Method and apparatus for high throughput cell-based assays for screening and diagnostics

(76) Inventors: John Maclaren Cassells, Cambridgeshire (GB); Paul Martin, Cambridgeshire (GB); Darrin Disley, Bedfordshire (GB)

Correspondence Address: David G Conlin, Edwards & Angell, Intellectual Property Practice Group, PO Box 9169, Boston, MA, 02209, US

(21) Appl. No.: 10/204,765

(22) PCT Filed: Feb. 23, 2001
(86) PCT No.: PCT/GB01/00804

(30) Foreign Application Priority Data
Feb. 25, 2000 (GB) 0004529.4

(51) Int. Cl. G01N 33/53; G01N 33/567; G06F 19/00; G01N 33/48; G01N 33/50; C12M 1/34; G01N 1/30

(52) U.S. Cl. 435/7.2; 702/21; 435/287.1; 435/40.5

(57) ABSTRACT

A method of performing an assay to detect and quantify changes in morphology or intracellular events in living or dead cells. The method comprises the step of presenting the cells on a surface for analysis in a single sample or array of samples and providing a means of fluorescently labelling cell structures, engulfed or associated particles or molecules contained on or within the cells. The cells are scanned with a detection system to obtain a linearseries of intensity values at intervals of 10 microns or less across each cell to produce line amplitude data in at least one emission band. A threshold algorithm is applied to determine the beginning and end of each feature on the line amplitude data, whereina feature is any perturbation from a determined background signal. The determined line amplitude data is processed for each feature for one or more emission bands to generate a value for at leastone of area specific intensity, peak intensity, half-width, half-width specific intensity, total intensity, peak intensity, peak ratio, inflection ratio, ID gaussian fit, 2D gaussian fit or a mathematical combination of these values.

This invention relates to cell based assays. The ability to characterise physiological changes in cells at a cellular or sub-cellular level is important in both drug discovery and clinical diagnostics. A successful approach to drug discovery has been the isolation of individual molecular interactions (for example protein:ligand interactions) implicated in a disease and the construction of assays to determine the activity of molecules in controlling or perturbing those interactions. High throughput screening involves testing very large numbers of compounds (perhaps a million) individually against such an assay in the search for compounds that may prove to be candidate drugs, or more commonly will give clues as to the chemical entities that could form the basis of a drug.

Drug discovery has become ever more complicated as researchers turn their attention to diseases that involve highly complex processes within the cell involving multiple molecular interactions as well as, in some cases, interaction with super-molecular physical structures such as membranes and organelles. Many new targets cannot be readily reduced to a cell-free assay of the interaction of only a small number of molecules. This may be because the full molecular interaction or signalling pathway is not known, the interacting molecules cannot be isolated in a functional form from the cell, there is no suitable method for constructing an assay from the components or that physical structures within the cell play a part in the process. The emergence of genomics has enabled researchers to probe disease mechanisms by introducing or controlling genes directly in the cell. Consequently, there is a need to be able to quantify changes in large numbers of individual cells or of processes within cells at speeds consistent with modern day drug discovery (of the order of 100,000 samples per day).

Researchers in drug discovery have for some time studied the effects of compounds and physiological conditions on whole cells as a means of discovering drugs that will control or influence these more complex processes. In the most common cell-based assays an aggregate signal is obtained for a large number of cells in a sample. The individuals cells themselves are not observed. Typical examples would be in some reporter gene assays. For example the expression of green fluorescent protein in cells can be determined by measuring the aggregate fluorescence of a whole well containing tens of thousands of cells with a low resolution detector. Similarly, β galactosidase expression is typically measured by analysing the cells and measuring the activity of the released enzyme in a luminescence assay whereby the luminescence of the whole well is measured.

Increasingly, researchers are interested in determining changes in cells that involve physical features or processes that are smaller than the cell itself. Examples include cell differentiation such as the growth of neurites on nerve cells, sub-cellular translocations such as the translocation of a transcription factor into the nucleus from the cytoplasm, changes in the cell through necrosis or apoptosis or the interaction of one cell with another such as phagocytosis of an apoptotic cell.

A similar requirement exists in cell diagnostics, where there is a requirement to detect changes in cells that are indicative of disease, such as differentiation (or lack of differentiation) of cancer cells, or polynucleation.

The conventional approach to studying morphological or physiological changes to a cell or cell structure is the use of microscopy. The sensitivity of microscopy is greatly enhanced by using fluorescent stains to label the features of interest. Microscopic examination of cells is inherently a slow technique. Enhancements in throughput have been obtained by the use of CCD cameras to capture images from a microscope and image analysis software to analyse and make measurements on cells. Camera-equipped microscopes and their derivatives have existed for many years. These are capable of capturing images of individual cells, processing the image and reporting differences in the cell due to changes in the cell itself or the localisation or relocation of fluorescent labels.

CCD cameras used in camera-equipped microscopes may have up to a million pixels. This is only sufficient, however, to capture an image of around 1 mm² with sufficient resolution to provide information on individual cells. Samples for examination are presented as wells in microscope slides or microtitre plates. The choice of microscope slides or microtitre plates is incidental as many manufacturers have long provided multi-well microscope slides (for example, Hendley-Essex, England) or special clear-bottomed plates with bottoms made of quartz, glass or plastic. Essentially, all the microscope-based imaging systems are limited to reading one well at a time because of the small scan area. It is obvious to anyone skilled in the art that the method of capturing images in a single well can be easily extended through repetition to capture images from multiple wells one at a time. Indeed, the microtitre plate is based on the footprint of four standard microscope slides and motorised XY stages for indexing samples from one slide to the next (such as those supplied by Prior, Cambridge, England), or from one well to the next, have been commercially available for microscopes for a considerable time. There is a large body of prior art concerned with the use of digital imaging to identify cells and to measure distribution of various fluorescent probes on or within cells. Some examples are Lee et al “A Digital Microscopy System for Rare-event Detection Using Fluorescent Probes”, Cytometry 10: 256-262, 1989; Mesker et al “Detection of Immunocytochemically Stained rare Events Using Image Analysis”, Cytometry 10:256-262, 1989; Ploem-Zaaijer J J. et al “Automated Image Cytometry for Detection of Rare, Viral Antigen-Positive Cells in Peripheral Blood”, Cytometry 15:199-206, 1994. WO 9411841 and WO 8702802 describe systems for cell analysis that could be developed or applied to high throughput screening. U.S. Pat. No. 5,989,835 describes the application of a system using a microscope objective and camera following the above prior art to a method of image analysis for measuring translocations.

It has also long been recognised that methods and reagents developed for staining cells or organelles for flow cytometry may be readily applied to solid phase cytometry (including image analysis) and reagents are readily available from a number of commercial sources. A significant benefit is that solid phase cytometry allows living, attached cells to be measured.

Automated or semi-automated systems based on camera equipped microscopes have proved to be very useful tools for secondary screening, histology, immunocytochemistry, cytometry and histopathology, but they have significant disadvantages for application to high throughput screening and diagnostics, where throughputs of 10,000 samples or more per eight hour period may be required.

The reasons for this are as follows. Firstly, a charge coupled device camera (CCDs) has finite resolution. The largest CCDs available today have around 1 million pixels, There is therefore a compromise between field of view and resolution. This means that typically the field of view is only 1 ruin² with resolution of 4 μm at best. This is only sufficient to obtain poorly resolved images of around 100 cells at once, which is insufficient for obtaining statistically significant results in some assay types. When culturing cells in microwells, it is common for cells to congregate in the corners of the wells. This means that, typically, a CCD imaging system will not observe the majority of cells in a well.

Reducing the field of view will increase the resolution to be sufficient to allow accurate measurement of the size and shape characteristics of cells, however that will further reduce the number of cells observed at once.

Secondly, a CCD imaging system can only image one well of a multi-well format (slides or microplates) at once. Therefore, the slide or plate has to be moved by one well at a time to cover all the wells in a slide or plate. This mechanical movement is relatively slow and further reduces throughput.

The sensitivity of CCDs is a trade off between integration time and sensitivity. CCD cameras are substantially less sensitive than photomultiplier tubes. For example, a photomultiplier tube can measure the light from a single photon, and in a scanning system it is possible to measure the emission from as low as 100 molecules of fluorescein in under 1 microsecond. To achieve detection at these low light levels, it is common to integrate with a CCD camera for of the order of 30 minutes or more. CCD based imaging systems are therefore insufficiently sensitive for making quantitative measurements at low light levels (for example labelled ligand bound to cell surface receptors on cells where expression is low, perhaps only 5,000 receptors per cell) where high speed is required. Even at moderate light levels, CCDs may take only one frame a second. CCD arrays are not capable of repeatedly scanning an area at rates fast enough to perform measurements of rapid transients or time resolved fluorescence techniques (nanosecond to microsecond sampling rates).

A further disadvantage of CCD based imaging systems is that each pixel can only detect one colour at a time. Multi-colour images may be obtained by using a filter wheel in front of the CCD array, and taking multiple frames with different filters. This slows down the reading time, and if the sample moves during measurement (as free cells are likely to do in liquid), or if a translocation of a dye happens rapidly (as with fast ion channel assays) the spectral information is lost. Multiple CCD arrays can be used to collect images in multiple colours, but it is not possible to achieve perfect pixel alignment between detectors, or to have true simultaneous multi wavelength detection. CCD arrays do not exhibit uniform sensitivity across the visible range. It is very important for background rejection at low signal levels that true simultaneous spectral measurements are made.

U.S. Pat. No 5,663,057 describes an apparatus and method for rapidly detecting micro-organisms on a solid support. Although the methods in this are aimed at counting micro-organisms, the methods may be used for automatically counting biological cells in general. This is confirmed by the use of the apparatus to detect and count mammalian cells, and to pick out rare events in a single scan of some thirty-one million cells (Mignon-Godefroy et al, Cytometry 27: 336-344 (1997)) on a glass or membrane surface.

According to the invention there is provided a method of performing an assay to detect and quantify changes in morphology or intracellular events in living or dead cells, the method comprising the steps of:

presenting the cells on a surface for analysis in a single sample or array of samples;

optionally, providing an environment for maintenance of living cells;

providing a means of fluorescently labelling cell structures, engulfed or associated particles or molecules contained on or within the cells;

scanning the cells with a detection system capable of illuminating the cells to excite fluorescence on or in the cells and obtaining a linear series of intensity values at intervals of 10 microns or less across each cell to produce line amplitude data in at least one emission band;

applying a threshold algorithm to determine the beginning and end of each feature on the line amplitude data, wherein a feature is any perturbation from a determined background signal;

processing the determined line amplitude data for each feature for one or more emission bands to generate a value for at least one of area specific intensity, peak intensity, half-width specific intensity, total intensity, peak intensity, peak ratios, inflection ratio, 1D gaussian fit, 2D gaussian fit or any mathematical combination of these values;

using at least one generated value or values in combination to determine if the feature detected is or is not a cell and furthermore to characterise each cell according to its morphological state and/or the presence of an intracellular process;

dividing the data into groups associated with defined regions of the surface where a group corresponds to a defined location in an array or an individual well and determining different populations of cells, or a shift in the characteristics of a population of cells in a defined area or sample.

The cells may be presented on any suitable surface such as a microscope slide or clear-bottomed microtitre plate. The cells may be presented dry or be immersed in a liquid for maintenance of live cells. The process may optionally be performed in a chamber having temperature, humidity and gas control (for example carbon dioxide enriched air) for maintenance of living cells. The cells, engulfed or associated particles or cell structures may be rendered fluorescent by any single or combination of incorporation of absorbance of a flourophore attached to a biological molecule (for example fluorescently-labelled ligands or antibodies), by incorporation or generation of a fluorescent protein (for example green fluorescent protein), from the generation of a fluorescent stain by enzyme action (for example viability stains), from a dye absorbed onto or transported into the cells (for example calcium channel dyes or membrane dyes), or incorporated into a larger cell through the engulfment of a smaller dyed cell.

The illuminating light may be generated by a laser beam. The received light is generated by fluorescence from the cells and is collected by one or more photosensitive detectors, such as photonmultiplier tubes. The laser beam is raster scanned across the sample to give a continuous amplitude reading at the detectors. The signal from the detectors is preferably sampled by a digital-to-analogue converter at a frequency such that the laser spot at the point of the sample will have progressed by less than its diameter between digital samples. Each line of the raster scan may be stepped by less than the diameter of the laser spot.

The detection system may alternatively be any system capable of generating line amplitude data such as an incandescent or discharge light source and CCD camera.

A corresponding apparatus is also provided.

The present invention provides a means of measuring changes in the morphology of cells or of processes within a cell at high speed and high sensitivity, and furthermore to measure these changes in multiple emission wavelengths simultaneously. The invention provides a means to measure the characteristics of every cell in multiple wells in one scan, typically as many as 60,000 cells, and to provide quantitative data on these cells. Morphological changes that can be detected and measured by the invention include, but are not limited to, mitosis, neurite outgrowth, cell surface changes and shrinkage due to necrosis/apoptosis. Intracellular processes that can be detected and measured by the invention include, but are not limited to, nuclear and organelle translocations, mitochondrial tracking and phagocytosis of cells or foreign bodies. It is a particular objective of the invention to gain information about morphological changes or intracellular processes from analysis of the line amplitudes alone and therefore without the need to construct or process an image. The invention also provides a means of characterising cells on the basis of features that are too small for the apparatus to fully resolve (for example, that are equal in size or smaller than the laser spot diameter).

Examples of the present invention will now be described with reference to the accompanying drawings, in which:

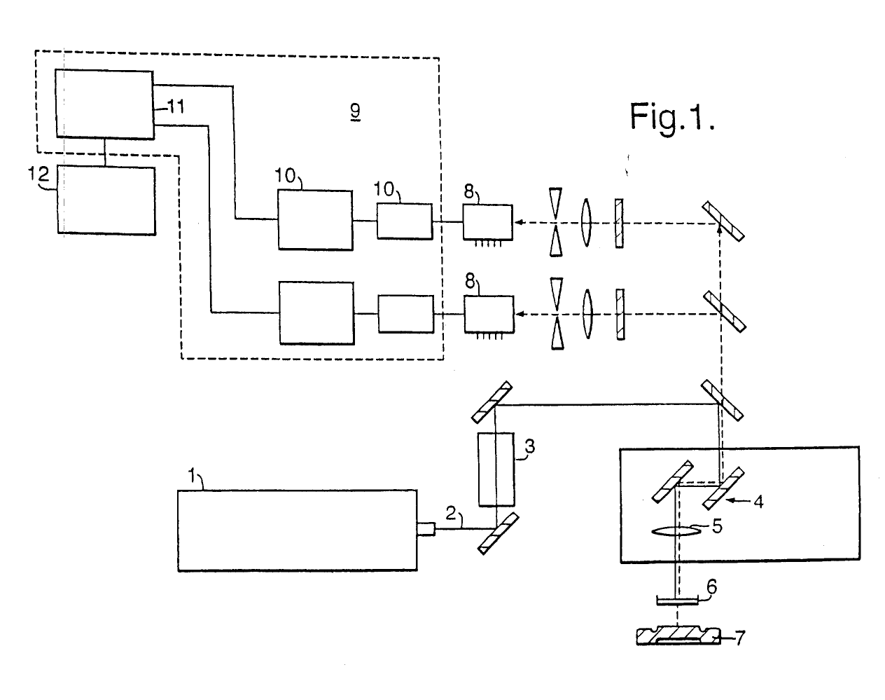

FIG. 1 is a schematic diagram of an apparatus that may be adapted to employ the present invention;

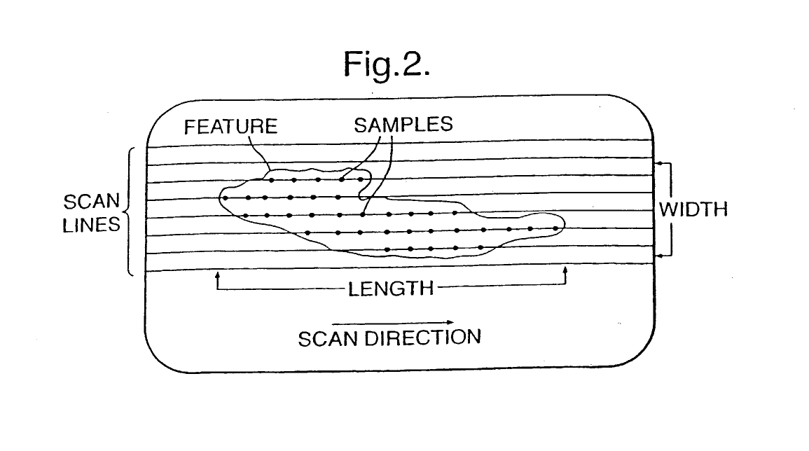

FIG. 2 is a diagram showing the scanning process of the apparatus of FIG. 1;

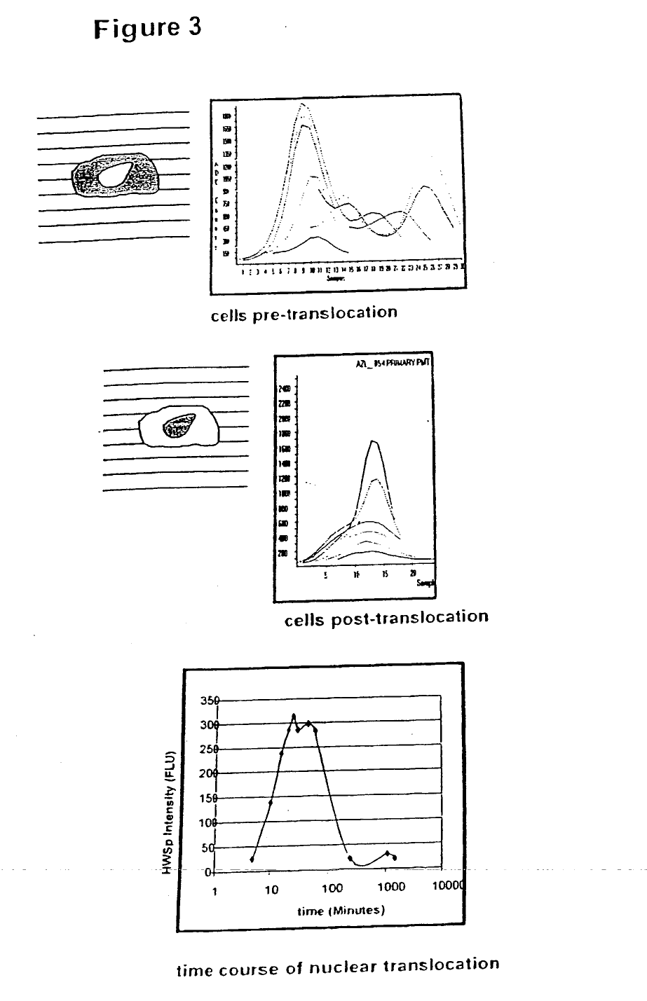

FIG. 3 is a series of graphs showing outputs of the present invention whilst performing a nuclear translocation assay;

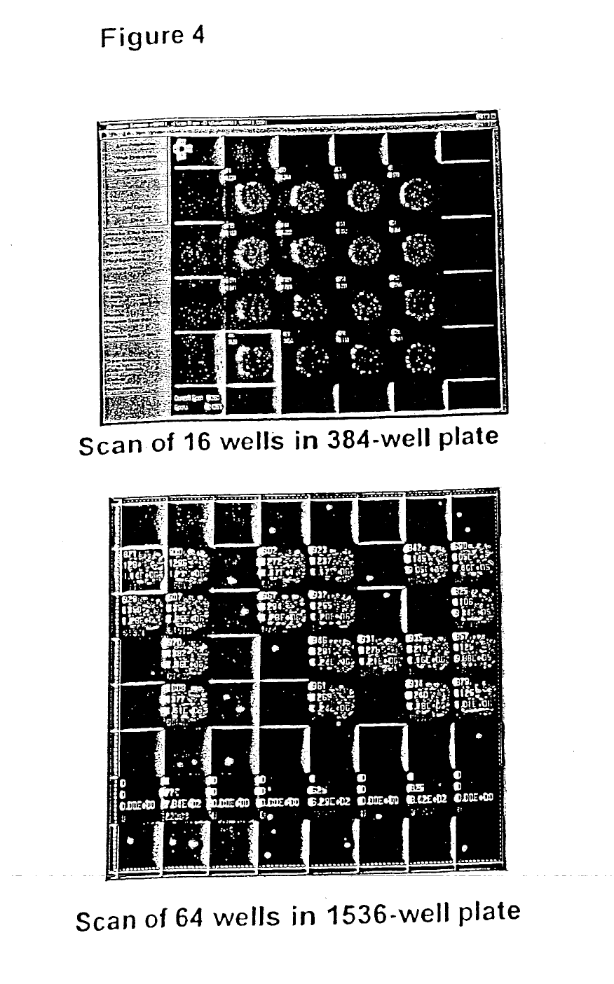

FIG. 4 is a diagram showing outputs of the invention during a simultaneous scan of multiple object wells;

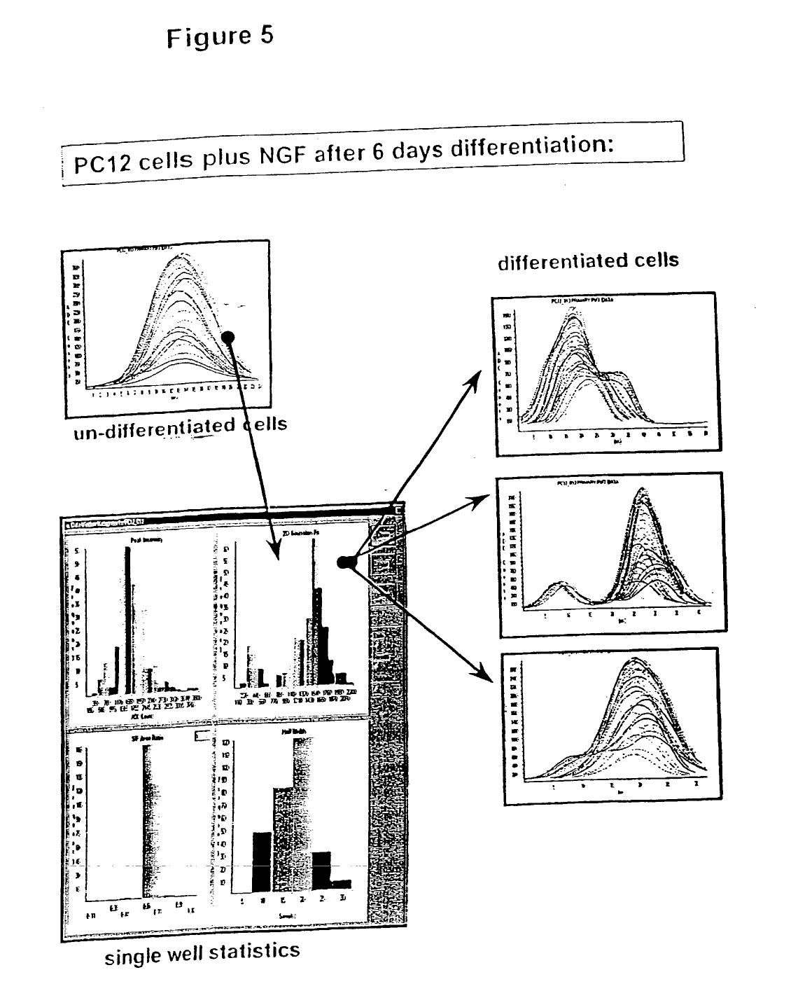

FIG. 5 shows the outputs of the invention from a cell differentiation;

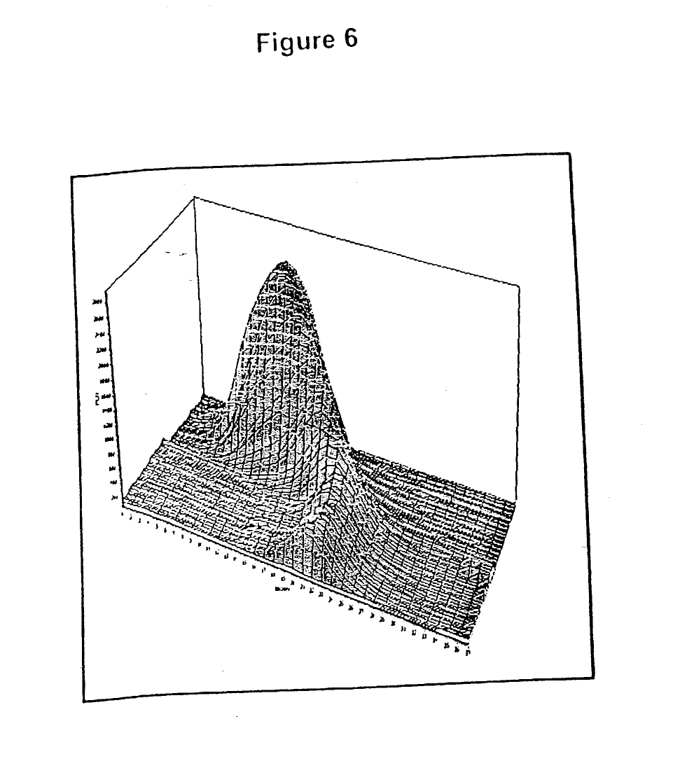

FIG. 6 is an example three dimensional representation generated by the invention; and

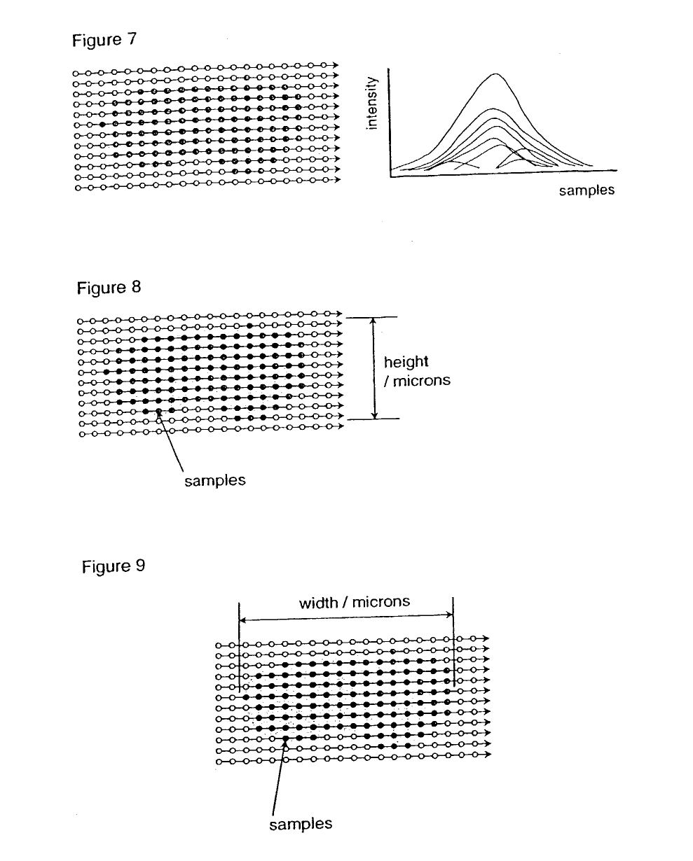

FIGS. 7 to 22 are schematic diagrams showing values that can be determined by the present invention.

FIG. 1 shows a diagram of the apparatus of U.S. Pat. No. 5,663,057, which is commercially available as the Chemscan RDI (Chemunex SA, France). This comprises a laser 1, which emits a laser beam 2. The laser light passes through a beam expander 3 and is scanned in X and Y by scan mirrors 4, and scan lens 5. The beam thus raster scans the sample holder 7. A focus tube 6 is used to align the sample holder. Emitted light passes through a pair of filter sets to two photomultiplier detectors 8. The signal from the detector is fed to an electronic system 9 which includes signal amplification 9, digital signal processing 11 and the resulting data is passed on to a computer 12 for further analysis. A device of this type can be adapted to provide an apparatus according to the invention. In a device according to the invention the photomultiplier preamplifiers 8 are uprated to provide higher gain, lower noise and a linear response.

Sampling electronics 9 provide 5 MHz sampling, and the control of the scanning mirrors 4 allows sub-micron line-to-line scan line increments to be achieved. An improved dynamic threshold based on rate of change of the PMT signals is in the software 12, together with a selectable “maximum feature length” setting to switch off the threshold in addition to the dynamic threshold.

Control and analysis software for the apparatus enables the computer to calculate parameters necessary for the method of the invention, together with advanced data handling to cope with the high volume of data obtained. The method of the invention may be performed on a variety of solid supports, 7. Practical supports 7 include membranes, microscope slides and microtitre plates. The method of the invention was initially performed on a glass microscope slide reading from above (a standard slide holder accessory is available). It is standard practice in image analysis and microscopy to employ an XY stage to allow a system to automatically index from one well to another for microscope slides and microtitre plates. This practice was followed by the addition of an XY stage to enable measurements to be made in microtitre plates automatically over a number of wells. When reading microtitre pates, the scan head was inverted to allow illumination and detection from below using clear bottomed microtitre plates.

The beam expander 3 in the invention is a motorised beam expander telescope to allow electronic control of focus. An additional proprietary system can be included to ensure the samples are in focus (not shown). The apparatus also has an additional third detector channel.

With the invention it is possible to detect and characterise cells and discrete objects smaller than the diameter of a laser spot used to illuminate them. FIG. 2 demonstrates how samples are taken, for example, every micron in the scan direction when scanning, and each scan line overlaps the previous one. The present invention teaches that it is possible to infer complex morphological or intracellular processes from the line amplitudes obtained from a scanning means without considering the outline or image of the cell or sub-cellular object being studied. A wide range of characteristics of cells and cell processes may be determined by applying measurements obtained from the data computed by the software. These measurements are described below.

Object Analysis

This phase of processing characterises the objects found during scanning. The raw data collected for an object consists of a collection of features. These are slices through the object, containing intensities measured across the object. Stored with each feature is the baseline of the noise prior to the feature (FIG. 7).

We calculate one or more of the following characteristics for an object: Height, Width, Total Intensity, Peak Intensity, Mean Intensity, Minimum Baseline, S/P Ratio, T/P Ratio, T/S Ratio, Half Width, Specific Intensity (Half Width) (also referred to as H/W Specific Intensity), Specific Intensity (Area Specific) (also referred to as Sample Specific Intensity), 1D Gaussian Fit, 2D Gaussian Fit, Peaks (also referred to as Peaks Ratio), Inflections (also referred to as Inflections Ratio), and Bound/Free.

Height

This is the height of the object in microns. This is the number of lines included in the object multiplied by the line spacing (vertical resolution), see FIG. 8.

Height=Number of Lines*Line Spacing

Width

This is the width of the object in microns. This is the number of samples from the left most feature to the end of the right most feature multiplied by the sample spacing (horizontal spacing), see FIG. 9.

Number of samples=Rightmost−Leftmost

Width=Number of samples*Sample spacing

Total Intensity

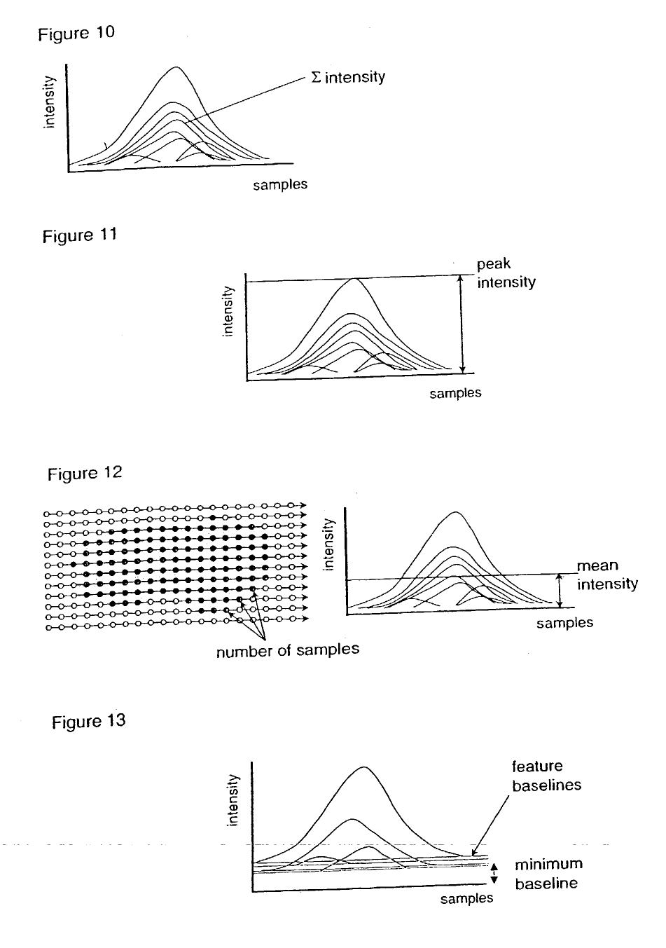

This is the sum of all the intensities measured inside the bounds of the object (see FIG. 10).

Total Intensity=Sum of all intensities found in each object.

Peak Intensity

This is the intensity of the greatest sample in the object (see FIG. 11).

Mean Intensity

This is the average of the intensities (see FIG. 12).

Mean Intensity=Total Intensity/Number of samples

Minimum Baseline (Primary, Secondary and Tertiary)

This is the lowest baseline of the features in the object at the point of thresholding (see FIG. 13).

S/P, TIP & T/S Ratio

These values allow the colour ratio of objects to be compared. Each label has a characteristic spectrum that will generate differing colour ratios (see FIG. 14).

Each of these ratios is calculated by summing the intensities for each channel and then dividing as appropriate. When calculating the sum of intensities the baseline value is subtracted giving a total intensity independent of the noise floor the object sits on.

Sum of primary intensities=Sum of intensities−baseline

Sum of secondary intensities=Sum of intensities−baseline

Sum of tertiary intensities=Sum of intensities−baseline

S/P=(Sum of secondary intensities/(Sum of primary intensities)

T/P=(Sum of tertiary intensities/(Sum of primary intensities)

T/S=(Sum of tertiary intensities/(Sum of secondary intensities)

Half Width

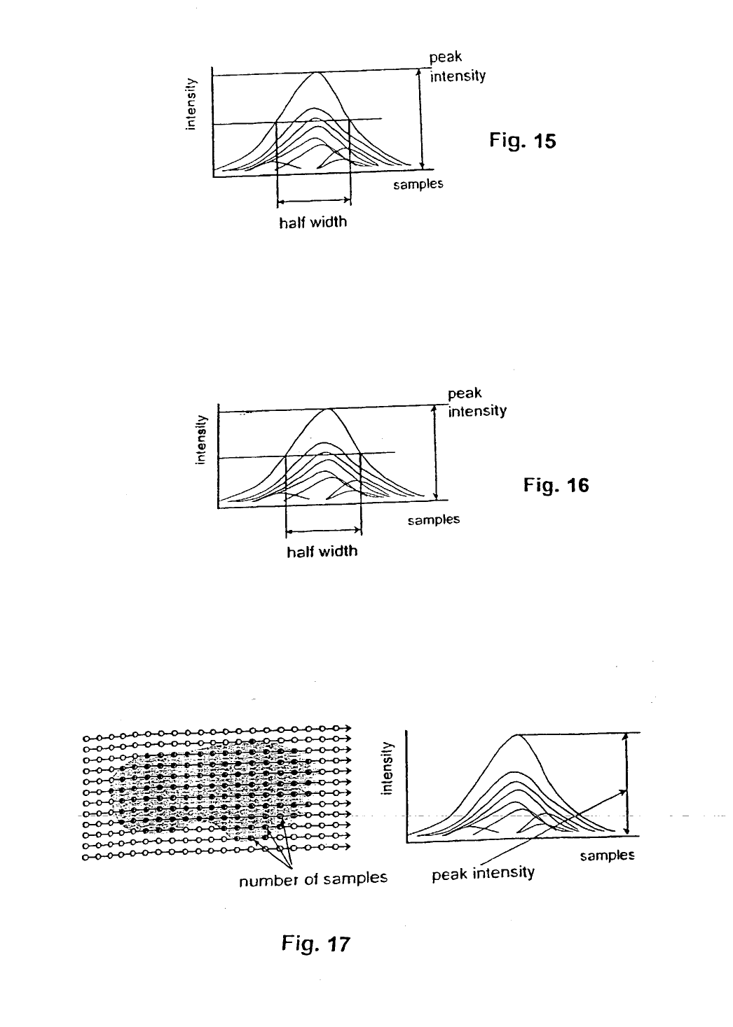

This is the width of the object at the point where the intensity drops to half of the peak (see FIG. 15). This may also be calculated for the dominant feature in the object.

Specific Intensity (Half Width)

This is the peak intensity of the object divided by its half width.

Specific intensity (Half Width)=Peak Intensity/Half Width

Specific Intensity (Area Specific)

This is the peak intensity of the object divided by the number of samples in the object as shown in FIG. 17.

Specific intensity (Area Specific)=Peak Intensity/Number of samples

1D Gaussian Fit

This is a Gaussian fit of the dominant feature (the one that contains the peak intensity) to an idealised Gaussian shape. The resulting value is scaled to the range 0 to 4095 where 0 indicates a perfect fit.

2D Gaussian Fit

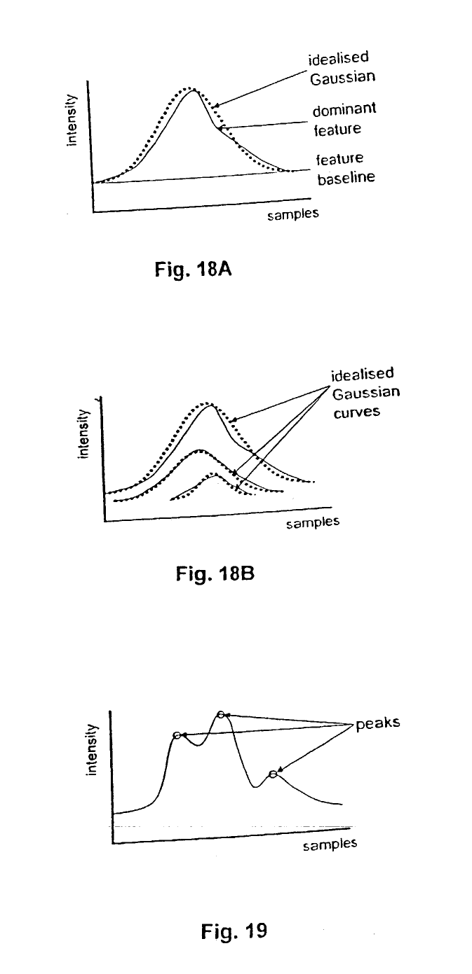

This is a Gaussian fit of the peaks of each feature within the object to an idealised Gaussian shape. The resulting value is scaled to the range 0 to 4095 where 0 indicates a perfect fit as shown in FIG. 18.

Peaks

This is the average number of peaks found within each feature in the object as shown in FIG. 19.

Peaks=Total number of peaks/number of lines

Inflections

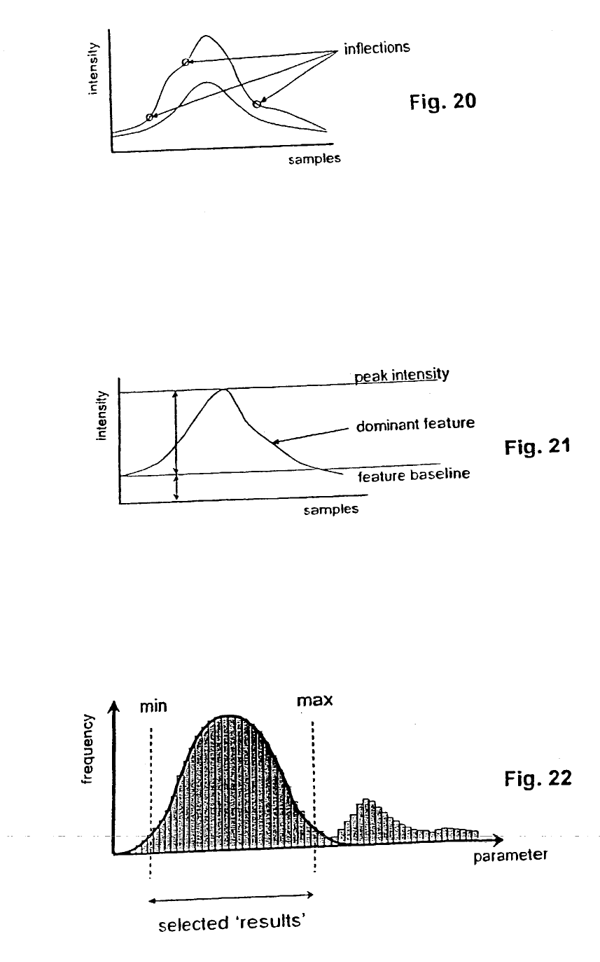

This is the average number of inflections found within each feature in the object as shown in FIG. 20.

Inflections=Total number of inflections/number of lines

Bound/Free

This is the peak intensity (minus baseline) divided by the baseline. This provides a measure of how much fluorescence has bound to an object or cell and how much remains unbound (FIG. 21).

Bound/Free=Peak intensity−Baseline/Baseline

Discrimination

This phase of processing classifies the objects found during scanning into two groups results and non results. Discrimination involves checking if an object's characteristics lie within specified bounds. If the object fails any of the bounds checks it is not classified as belonging to a defined classification. FIG. 22 shows a histogram of one parameter measured for all the objects found in a sample. Objects that fall within the left hand population are in this case classified as “results”. It can be seen that objects may also be classified as belonging to the right hand population.

The system and method of the invention allows discrimination to be performed on the following object characteristics: Height, Width, Peak Intensity, S/P Ratio, T/P Ratio, Half Width, Specific Intensity (Half Width) (also referred to as H/W Specific Intensity), Specific Intensity (Area Specific) (also referred to a Sample Specific Intensity), 2D Gaussian Fit, Peaks (also referred to as Peaks Ratio), and Inflections (also referred to as Inflections Ratio).

Well Analysis

The invention can also generate population histograms for all objects in a sample or well. Key characteristics of a well are:

Number Of Results

Mean Total Intensity

Mean Peak Intensity

Mean 2D Gaussian

Mean Specific Half Width Intensity

Mean peak ratio

Mean half width

Mean T/P ratio

Mean inflection ration

Mean S/P ratio

All of these characteristics are calculated from an aggregate of all the objects classed as results in the well by the discrimination process.

Number of Results

This is the number of objects which were classified as results by the discrimination process.

Mean Peak Intensity

This is the average peak intensity of all the result objects in the well.

Mean Total Intensity

This is the average total intensity of all the result objects in the well retainer.

Mean 2D Gaussian

This is the average 2D Gaussian value of all the result objects in the well.

Mean Specific Half Width Intensity

This is the average Specific Half Width Intensity of all the result objects in the well. It can be appreciated that the measurements described above can be further combined or refined, and that the raw data obtained by the method of the invention contains information that could be extracted by additional calculation.

The invention will now be described with reference to examples.

### EXAMPLE 1

Using the apparatus of the invention and employing a sampling rate of 5 MHz, a laser spot of 6 microns and a scan speed of 2 ms⁻¹ samples of an assay were taken every 0.4 microns. Low noise linear electronics provided a high quality amplitude signal which enabled small signal inflections to be accurately measured. The method of the invention was applied to the measurement of translocation of fluorescent molecules such as a flourescent protein or fluorescently-labelled protein from the cytoplasm into the nucleus. A translocated protein may also be visualised using a fluorescently-labelled antibody. FIG. 3 shows the line amplitudes of cells that have been scanned with the apparatus. The diagrams to the left of the line amplitude plots indicate the mode of scanning of the cells. It can be seen that cells pre-translocated (top plot) have a characteristic line amplitude shape that corresponds to the presence of fluorescent molecules in the body of the cell (cytoplasm) and a “hollow” in the signal corresponding to where the nucleus would be. The nucleus contains few fluorescent molecules. Post-translocated cells show a distinctly different line amplitude signal (middle plot) that corresponds to less bright cytoplasm and an intense peak corresponding to a labelled nucleus. An assay is performed by firstly setting discriminants to find and isolate cells on the basis if their line amplitudes and line-to-line correlation (not on the outline of an “image”). Specific measurements described previously can be combined to give a quantitative measurement of cell characteristics. In the case of the present example, the extent of translocation can be determined by measuring the specific intensity of the cells from the line amplitudes. A ratio of pre-translocated to post-translocated cells can readily be determined. The bottom plot on FIG. 3 shows how the time course of translocation was followed by the method of the invention using the mean half width specific intensity of the population as a measure of translocation. It is also possible to categorise cells as being in a pre-translocated or post-translocated state and thus to compute a ratio of respondent to non-respondent cells.

In fact, it is not necessary to obtain an accurate outline of a cell to perform the measurement. Throughput in an assay can be greatly increased by increasing the line-to-line spacing of the scanning just for it to be sufficient to identify that an object is likely to be a cell. The sub-cellular process (in this case translocation) can be measured from the line amplitude signals by considering the cell as a whole without recourse to isolating the signal from the nucleus from the signal from the cell. This example provides proof that, although the laser scanning system cannot perfectly resolve subcellular features smaller than the laser beam, the signals obtained contain enough amplitude information to make measurements of sub-cellular features and processes.

This technique, combined with the scanning of U.S. Pat. No. 5,663,057 enables much higher measurement speeds to be achieved. For a given scan speed the scan time is a determined by the line to line spacing. The apparatus of the invention can scan 400 mm² which corresponds to 16 wells of a 384-well microplate (FIG. 4) or 64 wells of a 1536-well plate at once, making it very much faster than microscope-based techniques which index from one well to the next. The data for 64 wells is collected at once and the results for each well is calculated by the computer. By increasing the line-to-line spacing it is possible to read 64 wells in under 2 minutes, giving a throughput potential of 60,000 to 100,000 assays per day matching the requirements of high throughput screening. Furthermore, it is critically important that a sufficient number of cells are measured to obtain good statistically-significant data. This is particularly important in high throughput screening where it is desirable to measure the activity of a compound in relation to tens of thousands of others. This requires good coefficients of variation (CV's) between replicate data sets. The present invention can scan typically 500-1,000 cells in every well (as opposed to only 100 for typical imaging methods) and also ensures that substantially all the cells in the well are detected (even in the corners). The high sensitivity of the PMT detectors allows the measurement of fluorescent molecules (such as reporters) some three orders of magnitude below that achievable with a camera running at moderate throughput.

It will also be appreciated that the present invention can provide simultaneous information in three or more emission wavelengths, and that further each emission wavelength may be processed independently by the method of the invention such that features or cells labelled with different labels may be independently measured to allow multiplexed assays or the determination of several features inside a cell at once.

### EXAMPLE 2

The method of the invention was applied to the measurement of neurite outgrowth from PC12 cells. The neurites are much smaller than the cells and the laser beam, being only one micron in diameter in some cases. FIG. 5 shows the method of the invention being applied to a population of PC12 cells to which nerve growth factor (NGF) has been added. After six days there were two populations of cells, one undifferentiated and the majority differentiated with neurite outgrowths. The extent of differentiation can be measured by considering the characteristic gaussian fit, multi-peaks ratio, sample width and number of inflections of the line amplitudes of the two populations. Any combination of these measurements from the line amplitudes may be used as a quantitative measure of differentiation. FIG. 5 (bottom) shows the clear separation of the two populations in the gaussian fit parameter histograms.

If the line-to-line spacing of the scan is sufficiently small it is also possible to produce a representation of each cell in 3D (FIG. 6). This provides confirmation of the ability of the method to characterise differentiation.

It should be noted that the line amplitudes and 3D plots are not exact replicas of the cell because the laser spot diameter is not much smaller than the cell itself (perhaps one third of the diameter of a small cell), and is much larger than small features such as neurites. This is not necessary for making measurements in an assay, so long as each individual type of cell and. fluorescent feature gives a consistent signal or signature. However, it is possible by Fourier de-convolution to provide a more accurate representation of the cells if this is required.

The method of the invention has been applied to determine morphological changes or intracellular processes in cells by the processing of line amplitudes alone, and furthermore, is able to indicate the likely presence of morphological features or sub-cellular translocations that are too small for the apparatus to fully resolve. Specific examples are the use of:

Half-width specific intensity and/or peak intensity of the brightest line amplitude plot across a cell to indicate a nuclear translocation or other translocation event,

the use of 1D gaussian and/or 2D gaussian shape of the line amplitudes to indicate cell differentiation such as neurite outgrowth.

The use of peak ratio and/or inflection ratio as a secondary measure of cell differentiation (it has been observed that PC12 cells show increased peak ratios and inflection ratio on differentiation when developing nuerites).

The use of peak intensity and/or peak ratio and/or inflection ratio in one emission channel (for example red emission) to indicate the presence of one or more engulfed fluorescently-stained cells (in this case red) in a macrophage stained a contrasting colour (for example green) by comparing the peak intensity and/or peak ratio and/or inflection ratio in one emission channel (for example red channel) with the peak intensity and/or peak ratio/ and or inflection ratio in another emission channel (for example the green channel). The ratio gives a measure of the number of cells engulfed per macrophage.

The use of peak ratio and/or half-width to count the number of cells undergoing mitosis in a population. Larger half-widths corresponds to cells undergoing mitosis. Higher peak ratio and/or inflection ratio indicate cells at advanced stages of division.

1. A method of performing an assay to detect and quantify changes in morphology or intracellular events in living or dead cells, the method comprising the steps of:
presenting the cells on a surface for analysis in a single sample or array of samples;
providing a means of fluorescently labelling cell structures, engulfed or associated particles or molecules contained on or within the cells;
scanning the cells with a detection system to illuminate the cells to excite fluorescence on or in the cells and obtaining a linear series of intensity values for light received therefrom at intervals of 10 microns or less across each cell to produce line amplitude data in at least one emission band;
applying a threshold algorithm to determine the beginning and end of each feature on the line amplitude data, wherein a feature is any perturbation from a determined background signal;
processing the determined line amplitude data for each feature for one or more emission bands to generate a value for at least one of area specific intensity, peak intensity, half-width, half-width specific intensity, total intensity, peak intensity, peak ratio, inflection ratio, 1D gaussian fit, 2D gaussian fit or a mathematical combination of these values; and
using at least one generated value or values in combination to determine if the feature detected is or is not a cell and furthermore to characterise each cell according to its morphological state and/or the presence of an intracellular process.

2. A method according to claim 1, wherein the received light is generated by fluorescence.

3. The method of claim 1 or claim 2, wherein the illuminating light is generated by a laser beam.

4. A method according to claim 1 or claim 2, wherein the illuminating light is generated by an incandescent light source, a inert-gas discharge lamp, or a metal vapour arc lamp.

5. A method according to claim 1, 2 or 3, wherein the received light is measured by one or more photonmultiplier tubes.

6. A method according to claim 1, 2 or 3, wherein the received light is measured by one or more photosensitive semiconductors.

7. A method according to any of claims 1 to 4, wherein the received light is measured by one or more charge coupled device (CCD) arrays.

8. A method according to claim 1 to 3, 5 or 6, wherein the laser scans the sample in a linear fashion, so that a continuous measurement of received light intensity can be provided by the detectors as line amplitude data.

9. A method according to claim 8, wherein the signal from the detector is digitally sampled at a rate equivalent to or less than the time taken for the laser spot to travel one laser spot diameter across the sample.

10. A method according to claim 7, wherein the digital signal from the CCD array is streamed out to provide line amplitude data.

11. A method according to any of the preceding claims, wherein more than one wavelength of light is received.

12. A method according to claim 11, wherein each received wavelength is processed separately.

13. A method according to any of the preceding claims wherein a fixed or dynamic threshold algorithm is applied to the line amplitude data to determine the beginning and end of a feature in the data.

14. A method according to any of the proceeding claims, wherein the determining means determines at least one of peak intensity, width, mean intensity, minimum baseline, S/P ratio, T/P ratio, T/S ratio, half width, number of peak ratio, inflection ratio, half-width specific intensity and a value representing the fit to an idealised gaussian shape for each feature detected in the line amplitudes.

15. A method according to any of the proceeding claims, wherein features found at a corresponding position in an adjacent scan line are associated in the data as objects such that the Y co-ordinate of the object or cell may be expressed as a function of the number of lines through the object or as an equivalent distance.

16. A method according to claim 15, wherein a 2D gaussian fit may be determined for a set of features on adjacent scan lines representing an object or cell.

17. A method according to claims 13 to 16, wherein the measurements are used to characterise cells as belonging to one population or another by means of a fingerprint comprising a set of minimum and maximum values for one or more of the calculated parameters from the line amplitudes, such fingerprints corresponding to differences in the morphology or the occurrence of intracellular events.

18. A method according to any of the preceding claims, wherein the half width specific intensity of the feature scan line displaying the highest amplitude is employed as a measure of the extent of a nuclear translocation of a fluorescent molecule from the cytoplasm into the nucleus of a cell.

19. A method according to any of the preceding claims, wherein the half width specific intensity of the feature scan line displaying the highest amplitude is employed as a measure of the extent of a translocation of a fluorescent molecule from the cytoplasm into an organelle or mitochondria within a cell.

20. A method according to any of the preceding claims, wherein the peak intensity of the feature scan line displaying the highest amplitude is employed as a measure of the extent of a nuclear translocation of a fluorescent molecule from the cytoplasm into the nucleus of a cell.

21. A method according to any of the preceding claims, wherein the peak ratio and/or inflection ratio and/or the 1D or 2D gaussian fits of the feature scan line displaying the highest amplitude is employed as a measure of the extent of general differentiation of a cell.

22. A method according to any of the preceding claims, wherein the peak ratio and/or inflection ratio and/or the 1D or 2D gaussian fits of the feature scan line displaying the highest amplitude is employed as a measure of the extent of differentiation of a cell growing nuerites.

23. A method according to any of the preceding claims wherein the peak ratio and/or inflection ratio and/or peak intensity of the feature scan lines for one emission band are compared with the peak ratio and/or inflection ratio and/or peak intensity of the feature scan lines for another emission band to provide a measure of the number of particles or cells engulfed by another cell.

24. A method according to any of the preceding claims wherein the peak ratio and/or inflection ratio and/or peak intensity of the feature scan lines for one emission band are compared with the peak ratio and/or inflection ratio and/or peak intensity of the feature scan lines for another emission band to provide a measure of the number of mitochondria inherited by a daughter cell.

25. A method according to any of the preceding claims wherein the peak ratio and/or inflection ratio and/or the 1D or 2D gaussian fits and/or the half-width and/or the width of the feature scan line displaying the highest amplitude is employed as a measure of apoptosis or necrosis.

26. A method according to any of the preceding claims wherein the peak ratio and/or inflection ratio and/or the 1D or 2D gaussian fits of the feature scan line displaying the highest amplitude is employed as a measure of the stage of cell division.

27. A method according to any of the proceeding claims where the cells being analysed are rendered fluorescent by any one or more of the incorporation of a fluorescent protein into a component of the cell, by the production of fluorescent proteins by the cell, by the use of fluorescent membrane dyes, by the use of fluorescent antibodies, by the use of fluorescent ligands, by the use of fluorescent nucleic acids, by the use of a fluoregenic enzyme substrates or by the use of ion-channel dyes.

28. A method according to any of the proceeding claims wherein the cells are presented on a microscope slide, microtitre plate or biochip.

29. A method according to any of the proceeding claims wherein an environment for maintenance of living cells is provided.

30. An apparatus for performing an assay to detect and quantify changes in morphology or intracellular events in living or dead cells, the apparatus comprising:
means for presenting the cells on a surface for analysis in a single sample or array of samples;
a detection system;
means for scanning the cells with the detection system to illuminate the cells to excite fluorescence on or in the cells and obtaining a linear series of intensity values for light received therefrom at intervals of 10 microns or less across each cell to produce line amplitude data in at least one emission band;
means for applying a threshold algorithm to determine the beginning and end of each feature on the line amplitude data, wherein a feature is any perturbation from a determined background signal;
means for processing the determined line amplitude data for each feature for one or more emission bands to generate a value for at least one of area specific intensity, peak intensity, half-width, half-width specific intensity, total intensity, peak intensity, peak ratio, inflection ratio, 1D gaussian fit, 2D gaussian fit or a mathematical combination of these values; and
means for using at least one generated value or values in combination to determine if the feature detected is or is not a cell and furthermore to characterise each cell according to its morphological state and/or the presence of an intracellular process.

31. The apparatus of claim 30, wherein the detecting means includes a laser beam.

32. An apparatus according to claim 30, wherein the detecting means includes an incandescent light source, a inert-gas discharge lamp, or a metal vapour arc lamp.

33. An apparatus according to any of claims 30 to 32 wherein the received light is measured by one or more photonmultiplier tubes.

34. An apparatus according to any of claims 30 to 32, wherein the received light is measured by one or more photosensitive semiconductors.

35. An apparatus according to any of claims 30 to 32, wherein the received light is measured by one or more charge coupled device (CCD) arrays.

36. An apparatus according to any of claims 30 to 35, arranged to receive more than one wavelength of light.

37. An apparatus according to claim 36, wherein each received wavelength is processed separately.
